(19) United States
(12) Patent Application Publication
(10) Pub. No.: US 2023/0068620 A1
(43) Pub. Date: Mar. 2, 2023

(54) Adjustment or Weighting of Blood Pressure in Response to One or More Biophysical or Environmental Conditions

(71) Applicant: Apple Inc., Cupertino, CA (US)

(72) Inventors: Wegene H. Tadele, San Francisco, CA (US); Albert Wang, San Francisco, CA (US); Motohide Hatanaka, Menlo Park, CA (US); Nicholas R. Trincia, San Francisco, CA (US); William K. Smith, San Francisco, CA (US)

(21) Appl. No.: 17/900,678

(22) Filed: Aug. 31, 2022

Related U.S. Application Data
(60) Provisional application No. 63/239,178, filed on Aug. 31, 2021.

(51) Int. Cl. A61B 5/021; G16H 40/67; A61B 5/00; A61B 5/1455

(52) U.S. Cl. CPC A61B 5/021; G16H 40/67; A61B 5/681; A61B 5/1455

(57) ABSTRACT

A wearable blood pressure measurement device includes a first set of one or more sensors, a second set of one or more sensors, and a processor. The processor is configured to obtain, from the first set of one or more sensors, a first set of one or more sensor outputs indicative of a blood pressure of a wearer of the wearable blood pressure measurement device. The processor is also configured to obtain, contemporaneously with obtaining the first set of one or more sensor outputs and from the second set of one or more sensors, a second set of one or more sensor outputs indicative of a set of one or more conditions under which the first set one or more sensor outputs is obtained.

### CROSS-REFERENCE TO RELATED APPLICATION

This application is a nonprovisional and claims the benefit of U.S.C. 35 § 119(e) of U.S. Provisional Patent Application No. 63/239,178, filed Aug. 31, 2022, the contents of which are incorporated hereby by reference as if fully disclosed herein.

### FIELD

The described embodiments generally relate to determining a health parameter (e.g., a blood pressure), a movement, or an activity of a wearer of a monitoring device (e.g., a blood pressure measurement device).

### BACKGROUND

A person may temporarily don a wearable device, such as a blood pressure cuff, and measure a parameter related to their health, such as a blood pressure. Typically, the wearable device is removed when the device is not actively being used to acquire a measurement. Often, the user may be required to wear the device in a particular way, sit in a particular position, be still, and not talk while the parameter is being measured.

### SUMMARY

Embodiments of the systems, devices, methods, and apparatus described in the present disclosure are directed to wearable electronic devices, and to systems of wearable and non-wearable electronic devices (e.g., handheld electronic devices, portable electronic devices, and/or semi-stationary electronic devices). The device(s) may be used to obtain a first set of one or more sensor outputs from a first set of one or more sensors, and use the sensor output(s) to determine a health parameter (e.g., a blood pressure) of a person that is wearing a wearable measurement device. Additionally or alternatively, the device(s) may infer a movement or activity of a person that is wearing a wearable measurement device. The device(s) may also be used to, contemporaneously with obtaining the first set of one or more sensor outputs, obtain a second set of one or more sensor outputs from a second set of one or more sensors. The second set of one or more sensor outputs may be used to adjust or weight the health parameter, or adjust or weight the inference regarding the user's movement or activity.

In a first aspect, the present disclosure describes a wearable blood pressure measurement device. The device may include a first set of one or more sensors, a second set of one or more sensors, and a processor. The processor may be configured to obtain, from the first set of one or more sensors, a first set of one or more sensor outputs indicative of a blood pressure of a wearer of the wearable blood pressure measurement device. The processor may also be configured to obtain, contemporaneously with obtaining the first set of one or more sensor outputs and from the second set of one or more sensors, a second set of one or more sensor outputs indicative of a set of one or more conditions under which the first set one or more sensor outputs is obtained.

In another aspect, the present disclosure describes an electronic device. The electronic device may include a wireless communications interface and a processor. The processor may be configured to obtain a first set of one or more sensor outputs indicative of a blood pressure of a user; obtain a second set of one or more sensor outputs indicative of a set of one or more conditions under which the first set one or more sensor outputs is obtained; determine the blood pressure of the user using the first set of one or more sensor outputs; and adjust or weight the blood pressure of the user using the second set of one or more sensor outputs. At least one sensor output of the first set of one or more sensor outputs or the second set of one or more sensor outputs may be obtained via the wireless communications interface.

In another aspect, the present disclosure describes a method of determining a blood pressure. The method may include obtaining, from a first set of one or more sensors in a first electronic device, a first set of one or more sensor outputs indicative of a blood pressure. The method may also include obtaining, contemporaneously with obtaining the first set of one or more sensor outputs and from a second set of one or more sensors, a second set of one or more sensor outputs indicative of a set of one or more conditions under which the first set one or more sensor outputs is obtained. At least one sensor in the second set of one or more sensors may be in a second electronic device. The method further includes determining the blood pressure of the user using the first set of one or more sensor outputs, and adjusting or weighting the blood pressure of the user using the second set of one or more sensor outputs.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example of a wearable blood pressure measurement device;

FIGS. 2A and 2B show an example wearable blood pressure measurement device worn on an arm of a user, with the arm of the user in different positions;

FIG. 3 illustrates how a vertical offset of a wearable blood pressure measurement device, from a shoulder of a user, may be determined;

FIG. 4 shows an example set of electronic devices worn or held by a user, and additional electronic devices near the user;

FIG. 5 shows an example method of determining a health parameter or characterizing an activity of a user; and

FIG. 6 shows an example electrical block diagram of an electronic device.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following description is not intended to limit the embodiments to one preferred embodiment. To the contrary, it is intended to cover alternatives, modifications, and equivalents as can be included within the spirit and scope of the described embodiments as defined by the appended claims.

Determining an accurate blood pressure typically requires a person to assume a particular posture, position, or other conditions. For example, a person may have to sit with their feet on the ground, with their back straight and the arm on which a blood pressure cuff is placed at heart level, while not talking. If a person departs from these guidelines, a blood pressure obtained by a blood pressure measurement device may be inaccurate. However, if a person's blood pressure is to be monitored periodically and continuously (e.g., at regular or irregular intervals throughout the day or night), instructing the person to repeatedly pause what they are doing and meet a set of conditions (e.g., conform to a particular posture or position) can be very intrusive and disruptive.

Described herein are systems, devices, methods, and apparatus that improve the user experience for an always on, and even temporarily donned, blood pressure measurement devices. The systems, devices, methods, and apparatus obtain sensor outputs from sensors other than those that are used to obtain a blood pressure, and adjust or weight a determined blood pressure for one or more conditions under which a blood pressure is determined. A blood pressure may be adjusted (e.g., corrected or compensated) for conditions such as, the various ways in which a person may position or wear a blood pressure cuff; movements that a person might make or activities that a person might engage in while sensors are operated to determine a blood pressure; a person's position or posture; a person's arm length or posing; a position or orientation of a person's arm with respect to their heart; whether a person is talking; a person's temperature; and so on. A blood pressure may also be weighted based on the above conditions. The weighting may indicate a confidence level in the blood pressure being accurate, or result in the blood pressure being weighted less (or even discarded) when used to determine an average blood pressure, making a diagnosis, or recommending next steps to be taken by the person, an application, or a medical professional.

In some cases, some or all of the sensors that are used to determine one or more conditions under which a blood pressure is obtained may be incorporated into a blood pressure measurement device. In some cases, some or all of the sensors that are used to determine the one or more conditions under which a blood pressure is obtained may be incorporated into other electronic devices, such as an electronic watch or health/fitness tracking device, a mobile phone (e.g., a smartphone) or other handheld portable device, one or more earbuds or a headset, a voice-controlled virtual assistant, a tablet computer, a laptop computer, a desktop computer, and a smart appliance (e.g., a refrigerator, a thermostat, etc.), and so on.

The sensors that are used to determine one or more conditions under which a blood pressure is obtained may determine or infer biophysical characteristics of a person whose blood pressure is being taken, determine whether the person is moving, infer what activity the person is engaged in, and so on.

The described systems, devices, methods, and apparatus can improve a person's experience while their blood pressure is being monitored—especially in a periodic and continuous blood pressure measurement scenario—by providing fewer interruptions to the person, by providing more accurate blood pressures, and so on.

The techniques used by the described systems, devices, methods, and apparatus can be extrapolated for use in periodically and continuously measuring other health parameters (e.g., a blood oxygen saturation, a heart rate, an electrocardiogram, a respiratory condition, a temperature, and so on), or periodically and continuously inferring a person's movements or activity.

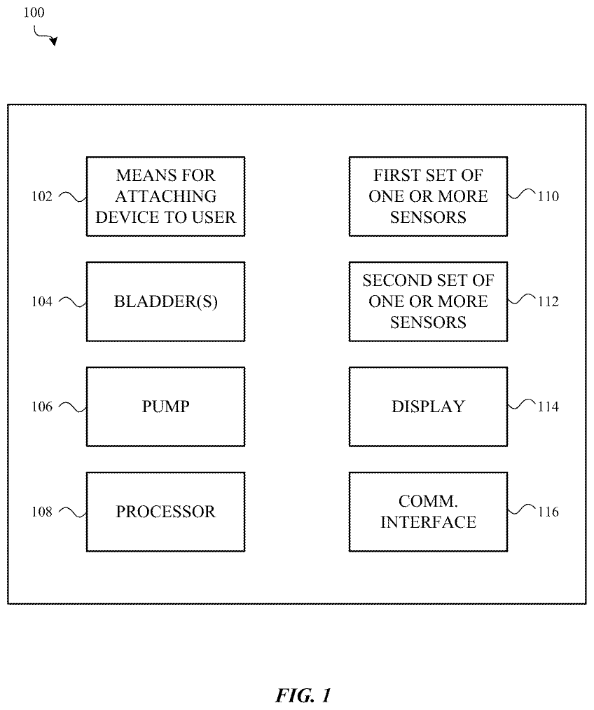

Described herein with reference to FIGS. 1-6 are systems, devices, methods, and apparatus that, in some cases, improve the determination of a health parameter (e.g., blood pressure), motion, or activity of a person wearing a wearable measurement device (e.g., a wearable blood pressure measurement device).

Directional terminology, such as “top”, “bottom”, “upper”, “lower”, “front”, “back”, “over”, “under”, “above”, “below”, “left”, or “right” is used with reference to the orientation of some of the components in some of the figures described below. Because components in various embodiments can be positioned in a number of different orientations, directional terminology is used for purposes of illustration only and is usually not limiting. The directional terminology is intended to be construed broadly, and therefore should not be interpreted to preclude components being oriented in different ways. Also, as used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

FIG. 1 shows an example of a wearable blood pressure measurement device 100. In some embodiments, the device 100 may be configured to be worn on an arm. In some embodiments, the device 100 may be configured to be worn on a wrist, or on another body part of a person whose blood pressure is being measured (i.e., a body part of a wearer of the device 100).

The device 100 may include a means 102 for attaching the device 100 to a body part, such as a cuff (i.e., a blood pressure cuff), a band, or a housing. By way of example, FIG. 1 shows the means 102 for attaching the device 100 to a user to be a cuff. In some embodiments, a cuff, band, housing, or other portion of the device 100 may house or otherwise be attached to one or more bladders 104, and the device 100 may include a pump 106 that expands and contracts the bladder(s) 104 (e.g., inflates and deflates the bladder(s) 104) under control of a processor 108 (or other control circuit or control system). When obtaining sensor outputs for the purpose of determining a blood pressure, the processor 108 may operate the pump 106 to temporarily expand and then contract the bladder(s) 104, such that a flow of blood through a portion of a person's circulatory system is temporarily occluded between expansion and contraction of the bladder(s) 104. In some embodiments, one or more pressure measurements, capacitive strain measurements, capacitive displacement measurements, optical measurements, or other types of measurements may be obtained while the bladders(s) 104 are expanding, expanded, or contracting, and the measurements may be used to determine a blood pressure. In alternative embodiments, the device 100 may or may not include the bladder(s) 104 and pump 106, and the processor 108 may determine a blood pressure from optical measurements, pulse transit time (PTT) measurements, or other types of measurements.

The device 100 may include a first set of one or more sensors 110 and a second set of one or more sensors 112. The processor 108 may obtain a first set of one or more sensor outputs from the first set of one or more sensors 110 and, contemporaneously with obtaining the first set of one or more sensor outputs, obtain a second set of one or more sensor outputs from the second set of one or more sensors 112. The first set of one or more sensor outputs may be indicative of a blood pressure of a wearer of the device 100. The second set of one or more sensor outputs may be indicative of a set of one or more conditions under which the first set one or more sensor outputs is obtained. When the device 100 includes one or more bladders 104 that can be expanded to temporarily occlude blood flow through a portion of a person's circulatory system, the processor 108 may operate a pump to expand and then contract the bladder(s), and obtain the first and second sets of measurements while the bladder(s) 104 are expanding, expanded, or contracting. As used herein, “contemporaneously” obtained sensor outputs are sensor outputs that are obtained at or about the same time (e.g., while a cuff is expanding, expanded, or contracting, or at particular synchronized or alternating times while the cuff is expanding, expanded, or contracting).

The first set of one or more sensors 110 may include any type of sensor that can be used alone, or in combination with other sensors, to measure a blood pressure. For example, the first set of one or more sensors may include one or more pressure sensors, strain sensors, capacitive sensors, or optical sensors.

The second set of one or more sensors may include, for example, acoustic sensors (including, in some cases, acoustic transducers that emit and receive ultrasonic pulses, i.e., inaudible mechanical waveforms), optical sensors (including, in some cases, active optical sensors that emit and receive optical pulses, such as infrared (IR) pulses), timers, time of flight sensors, temperature sensors, inertial measurement units (IMUs), and so on.

In some embodiments, the processor 108 may use the first set of one or more sensor outputs to determine a blood pressure of a wearer of the device 100. Subsequently to determining the blood pressure, or as part of the process of determining the blood pressure, the processor 108 may use the second set of one or more sensor outputs to adjust the blood pressure for the set of one or more conditions. The processor 108 may also or alternatively use the second set of one or more sensor outputs to weight (e.g., determine a confidence level for) the blood pressure.

The set of one or more conditions for which the blood pressure is adjusted may include, for example, a position of the device 100 on a body part (e.g., a position of a blood pressure cuff on an arm), a position of the device 100 relative to another body part (e.g., a position of a blood pressure cuff relative to the heart of a wearer of the device 100), a length of a body part, a position or orientation of a body part, relative positions of body parts, a position or posture of a wearer of the device 100, a temperature of a wearer of the device 100, an activity of a wearer of the device 100, and so on.

In some embodiments, the device 100 may include a display 114 and/or a wired or wireless communications interface 116 (e.g., a Wi-Fi or Bluetooth® interface). The processor 108 may cause a determined and adjusted blood pressure to be displayed on the display 114 and/or transmitted to a remote device via the communications interface 116. Additionally or alternatively, the processor 108 may cause the first and second sets of one or more sensor outputs to be transmitted to a remote device via the communications interface 116, and the remote device may determine and adjust the blood pressure of the wearer, save the sensor outputs and/or adjusted blood pressure, or transmit the adjusted blood pressure and/or sensor outputs to an additional device for determining and adjusting the blood pressure of the wearer and/or saving the sensor outputs and/or adjusted blood pressure.

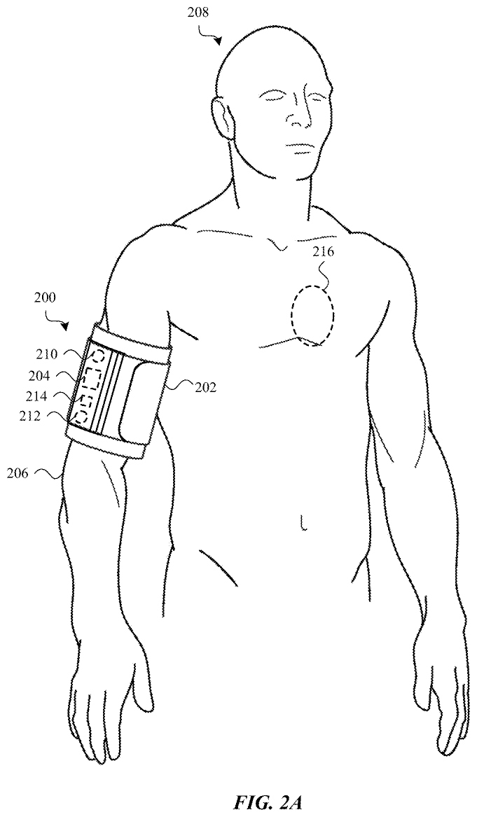

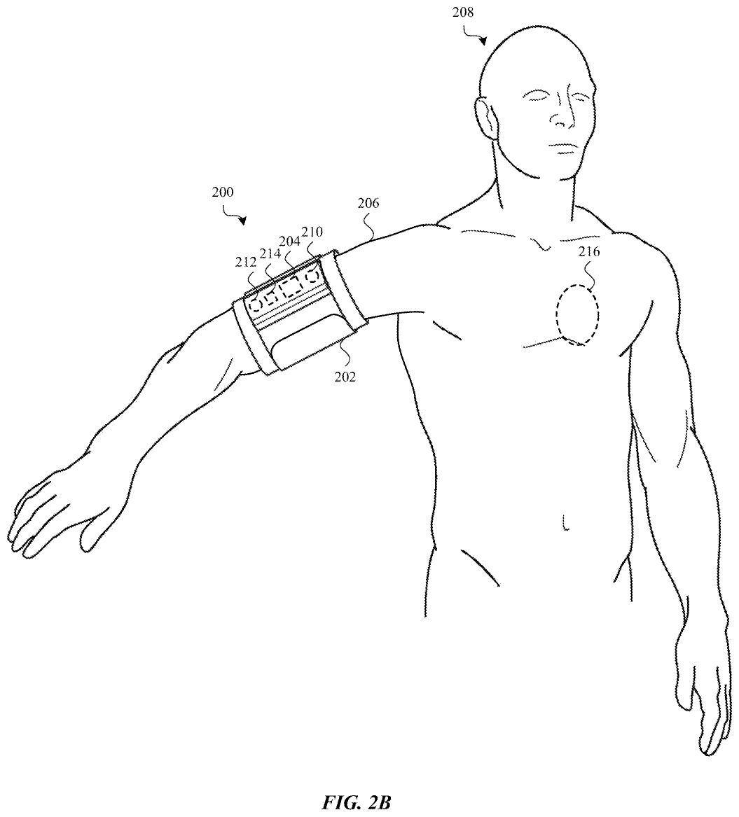

FIGS. 2A and 2B show an example wearable blood pressure measurement device 200 worn on an arm of a user. In some embodiments, the device 200 may be constructed similarly to the wearable blood pressure measurement device described with reference to FIG. 1 and include a means 202 for attaching the device 200 to a body part, an optional one or more bladders, an optional pump, a processor 204, a first set of one or more sensors, a second set of one or more sensors, an optional display, and an optional communications interface. By way of example, the device is shown to be wearable on an arm 206 of a user 208, and the means 202 is shown to include an inflatable cuff. FIG. 2A shows the arm 206 of the user 208 in a first position, and FIG. 2B shows the arm 206 of the user 208 in a second position.

In some examples, the second set of one or more sensors may include a timer associated with at least one acoustic transducer 210 or 212. Each of the acoustic transducer(s) 210, 212 may have a known position with respect to the device 200, such as a known position with respect to a blood pressure cuff of the device 200. The processor 204 may cause the at least one acoustic transducer 210, 212 to emit an ultrasonic pulse and listen for at least one echo of the ultrasonic pulse. The acoustic transducer 210 may emit an ultrasonic pulse toward a shoulder of a wearer of the device 200 and listen for at least one echo from the shoulder. The acoustic transducer 212 may emit an ultrasonic pulse toward a hand, elbow, or elbow pit of the wearer and listen for at least one echo from the hand, elbow, or elbow pit. One or more timers (e.g., a timer associated with both acoustic transducers or individual timers associated with respective individual acoustic transducers) may record a time of emission of an ultrasonic pulse and a time of receipt of at least one echo of the ultrasonic pulse (e.g., an echo occurring as a result of the ultrasonic pulse reflecting off of the wearer's shoulder, hand, elbow, or elbow pit). The processor 204 may obtain the second set of one or more sensor outputs by obtaining a time of emission of an ultrasonic pulse and a time of receipt of at least one echo of the ultrasonic pulse. Using the emission times and echo receipt time(s), the processor 204 may determine a position of the blood pressure measurement device 200 on the wearer of the wearable blood pressure measurement device 200 (e.g., by determining a roundtrip time (i.e., time of flight) of an ultrasonic pulse from a known acoustic transducer location on the device 200 to a feature (e.g., shoulder, hand, elbow, or elbow pit) of a user, and using the speed of the ultrasonic pulse to determine a distance to the shoulder, hand, elbow, or elbow pit). In some cases, the determined position may be a position of a blood pressure cuff of the device 200 on an arm of the wearer. The processor 204 may then use the determined position of the device 200 to adjust the wearer's blood pressure based on where the device 200 is worn on the wearer's arm. In some cases, such as when the processor obtains sensor outputs indicating times of emission and times of receipt of ultrasonic pulses transmitted toward both a wearer's shoulder and hand (and/or elbow or elbow pit), the processor 204 may also determine the length of the user's arm 206, or the lengths of portions of the user's arm 206. When the device 200 includes the display, the processor 204 may cause the adjusted blood pressure to be displayed on the display. When the device 200 includes the communications interface, the processor 204 may transmit the adjusted blood pressure to a remote device, or the processor 204 may not determine a blood pressure or an adjusted blood pressure and, instead, transmit the sensor outputs obtained from the first and second sets or one or more sensors to a remote device.

In some examples, the second set of one or more sensors may additionally or alternatively include a timer associated with at least one active optical sensor 210 or 212 (i.e., an electro-optical device including an optical emitter and an optical receiver). Each of the active optical sensor(s) 210, 212 may have a known position with respect to the device 200, such as a known position with respect to a blood pressure cuff of the device 200. The processor 204 may cause the at least one active optical sensor 210, 212 to emit an optical pulse and monitor for at least one reflection of the optical pulse. The active optical sensor 210 may emit an optical pulse toward a shoulder of a wearer of the device 200 and monitor for at least one reflection from the shoulder. The active optical sensor 212 may emit an optical pulse toward a hand, elbow, or elbow pit of the wearer and monitor for at least one reflection from the hand, elbow, or elbow pit. One or more timers (e.g., a timer associated with both active optical sensors or individual timers associated with respective individual active optical sensors) may record a time of emission of an optical pulse and a time of receipt of at least one reflection of the optical pulse (e.g., a reflection occurring as a result of the optical pulse reflecting off of the wearer's shoulder, hand, elbow, or elbow pit). The processor 204 may obtain the second set of one or more sensor outputs by obtaining a time of emission of an optical pulse and a time of receipt of at least one reflection of the optical pulse. Using the emission times and reflection receipt time(s), the processor 204 may determine a position of the blood pressure measurement device 200 on the wearer of the wearable blood pressure measurement device 200 (e.g., by determining a roundtrip time (i.e., time of flight) of an optical pulse from a known acoustic transducer location on the device 200 to a feature (e.g., shoulder, hand, elbow, or elbow pit) of a user, and using the speed of light to determine a distance to the shoulder, hand, elbow, or elbow pit). In some cases, the determined position may be a position of a blood pressure cuff of the device 200 on an arm of the wearer. The processor 204 may then use the determined position of the device to adjust the wearer's blood pressure based on where the device 200 is worn on the wearer's arm. In some cases, such as when the processor obtains sensor outputs indicating times of emission and times of receipt of optical pulses transmitted toward both a wearer's shoulder and hand (and/or elbow or elbow pit), the processor 204 may also determine the length of the user's arm 206, or the lengths of portions of the user's arm 206. When the device 200 includes the display, the processor 204 may cause the adjusted blood pressure to be displayed on the display. When the device 200 includes the communications interface, the processor 204 may transmit the adjusted blood pressure to a remote device, or the processor 204 may not determine a blood pressure or an adjusted blood pressure and, instead, transmit the sensor outputs obtained from the first and second sets or one or more sensors to a remote device.

If the device 200 were to alternatively be configured as a wrist-worn device, an acoustic transducer or active optical sensor may emit an ultrasonic or optical pulse toward a wearer's upper arm or elbow, or toward a wearer's shoulder, and determine a length of a wearer's lower arm or entire arm in response to acoustic/optical pulse emission and echo/reflection receipt times.

In some embodiments, the second set of one or more sensors may additionally or alternatively include an IMU 214 (e.g., an accelerometer or gyroscope), and the second set of one or more sensor outputs may include a gravity angle. In these embodiments, the processor 204 may determine the gravity angle using the output of the IMU 214 (e.g., by simply obtaining the output of the IMU 214, or by deriving the gravity angle from the output of the IMU 214). The processor may then determine, using the gravity angle, at least one of: an orientation of the wearable blood pressure measurement device 200 (e.g., is the device 200 oriented such that a portion of the arm 206, or another body part on which the device 200 is worn, would have to be oriented vertically, horizontally, or in some other position); a position or orientation of a body part on which the wearable blood pressure measurement device 200 is worn (e.g., as inferred by the orientation of the device 200); or a position or orientation of the wearable blood pressure measurement device with respect to a torso or heart of a wearer of the wearable blood pressure measurement device 200. In some cases, the processor 204 may use the output of the IMU 214, in combination with a determined position of the device 200 on an arm and/or length of an arm or arm portion(s), to infer more specific details about the position or orientation of a user's arm, device 100, posture, or activity. The processor 204 may use a sequence of determined gravity angles to determine a movement or activity being performed by the user 208. The processor 204 may use a determined gravity angle, sequence of determined gravity angles, or information determined therefrom, to adjust the wearer's blood pressure based on an orientation of the wearable blood pressure measurement device 200; a position or orientation of a body part on which the wearable blood pressure measurement device is worn; a position or orientation of the wearable blood pressure measurement device 200 with respect to a torso or heart of a wearer of the wearable blood pressure measurement device 200; or a movement or activity of the user 208. When the device 200 includes the display, the processor 204 may cause the adjusted blood pressure to be displayed on the display. When the device 200 includes the communications interface, the processor 204 may transmit the adjusted blood pressure to a remote device, or the processor 204 may not determine a blood pressure or an adjusted blood pressure and, instead, transmit the sensor outputs obtained from the first and second sets or one or more sensors, including the IMU sensor outputs, to a remote device.

Alternatively or additionally to adjusting a blood pressure using sensor outputs obtained from the second set of one or more sensors, the processor 204, or a remote device that receives sensor outputs from the device 200 via the communications interface, may use the sensor outputs obtained from the second set of one or more sensors to weight (e.g., determine a confidence level for) a determined blood pressure. For example, a determined position or orientation of the device 200 or the user 208, or a movement or activity of the user 208, may suggest that a determined blood pressure is more or less accurate. When a user's blood pressure is determined periodically over a period of time, particular positions or orientations of the device or the user 208, or particular movements or activities of the user 208, may suggest that particular blood pressures should be given more or less weight when determining an average blood pressure or determining whether to flag the user's blood pressure as being within or outside of a defined thresholds.

FIG. 2A shows the arm 206 of the user 208 extending in a more or less vertical (or downward) position. In this position, the user's hand and elbow, as well as the blood pressure cuff of the device 200, are below (or lower than) the user's heart 216. In contrast, FIG. 2B shows the arm 206 of the user 208 extending in a more or less horizontal (or outward) position. In this position, the user's hand and elbow, as well as the blood pressure cuff of the device 200, are at about a same elevation as the user's heart 216. These inferences assume, however, that the user 208 is standing or sitting in an upright position. If the user 208 were instead lying flat, different inferences may be drawn.

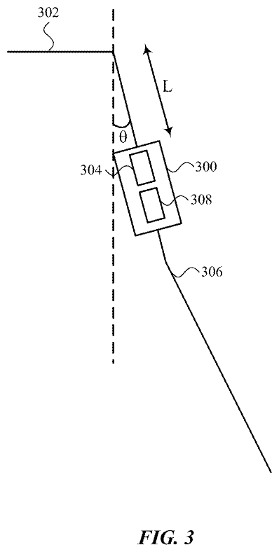

FIG. 3 illustrates how a vertical offset of a wearable blood pressure measurement device 300, from a shoulder 302 of a user (e.g., a wearer), may be determined. As shown, the device 300, or an acoustic transducer 304 or active optical sensor thereof, may be positioned on an arm 306 at a length L from the shoulder 302. An IMU 308 of the device 300 may provide a gravity angle, θ, or one or more sensor outputs from which the gravity angle, θ, may be determined. The gravity angle represents a departure of an arm position (or arm portion position) from a true vertical orientation (i.e., a direction of gravitational pull).

Using a simple model of the arm 306 as a pivot assembly, and basic trigonometry, the vertical offset of the device 300 from the shoulder 302 may be determined as

Vertical Offset=L cos θ

The vertical offset of the device 300 from the shoulder 302 can be used to estimate the force of gravity on the arm 306, which force can create a pressure differential between a blood pressure determined from arm measurements and a blood pressure of a person's torso. Determining the vertical offset of the device 300 from the shoulder 302 enables a blood pressure determined from arm measurements to be adjusted for the pressure differential.

If a person is wearing an electronic device containing an IMU on their wrist, or holding an electronic device containing an IMU in their hand, the IMU of the wrist-worn or handheld device may be provide additional sensor outputs (e.g., an additional gravity angle) that can be used, in conjunction with the model of the arm 306, to determine the position and/or orientation of a person's forearm, thereby enabling a determination of whether a person's arm 306 is bent or straight. Determining the lengths and orientations of a user's upper arm and forearm also provides an inference as to whether a user is sitting, standing, or lying down—especially if supplemented with an IMU output of a head-worn device (e.g., an IMU in an earbud or a headset, or multiple IMUs in a set of earbuds or headset).

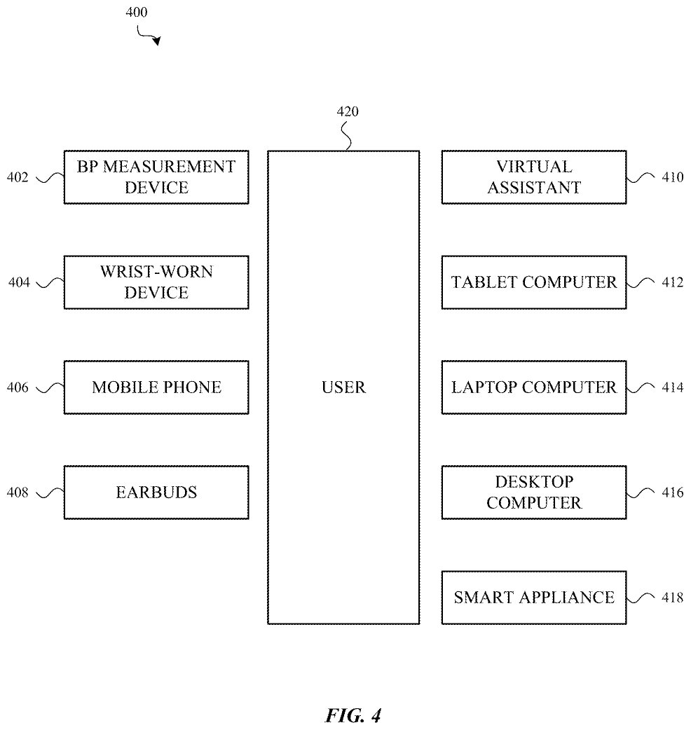

FIG. 4 shows an example set of electronic devices 400 that may be worn or held by a user 420, and additional electronic devices that are near the user 420. By way of example, the devices include a blood pressure (BP) measurement device 402 (e.g., a device worn on an arm or wrist of the user 420), an electronic watch or health/fitness tracking device 404, a mobile phone 406 (e.g., a smartphone) or other handheld portable device, one or more earbuds 408 or a headset, a voice-controlled virtual assistant 410, a tablet computer 412, a laptop computer 414, a desktop computer 416, and a smart appliance 418 (e.g., a refrigerator, a thermostat, etc.), and so on. All of the electronic devices 400 may communicate with each other, with a subset of one or more of the electronic devices 400, or with a remote electronic device, using one or more wired or wireless communication interfaces of the electronic devices 400.

In some embodiments, the blood pressure measurement device 402 may obtain (e.g., from a first set of one or more sensors) a first set of one or more sensor outputs indicative of a blood pressure of the user 420, as described herein. The blood pressure measurement device 402 and/or one of the other electronic devices 404-418, and in some cases all of the electronic devices 402-418, may individually or collectively obtain (e.g., from a second set of one or more sensors) a second set of one or more sensor outputs indicative of a set of one or more conditions under which the first set one or more sensor outputs is obtained. The first and second sets of one or more sensor outputs, and/or a blood pressure determined from the first set of one or more sensor outputs, and/or one or more determinations regarding the set of one or more conditions, may then be used by a single one of the electronic devices 400, or by a distributed set of the electronic devices 400, or by a remote device in communication with one or more of the electronic devices. One or more of the electronic devices 402-418 (or a remote electronic device) may use the first set of one or more sensor outputs to determine a blood pressure of the user 420. The same one or more electronic devices 402-418 (or a remote electronic device), or a different one or more of the electronic devices 402-418 (or a remote electronic device), may adjust or weight (e.g., determine a confidence level for) the blood pressure of the user 420 using the second set of one or more sensor outputs. Regardless of which electronic device(s) 402-418 (or a remote electronic device) determines, adjusts, and/or weights the blood pressure, the electronic device(s) may include a wired or wireless communications interface that can be used to obtain or transmit at least one sensor output of the first or second set of one or more sensor outputs.

In some examples, the second set of one or more sensor outputs may include a time of emission of an ultrasonic pulse emitted by at least one acoustic transducer in the wearable blood pressure measurement device 402, and at least one time of receipt of at least one echo of the ultrasonic pulse. In these examples, a processor of the wearable blood pressure measurement device 402, or a processor of any of the electronic devices 400 (or a remote electronic device) that obtains the first set of one or more sensor outputs (or a blood pressure) and/or the second set of one or more sensor outputs from the wearable blood pressure measurement device 402, may use the time of emission of the ultrasonic pulse and the at least one time of receipt of the at least one echo to determine a position of the wearable blood pressure measurement device 402 on the wearer of the wearable blood pressure measurement device 402. The same processor, or a processor of another one of the electronic devices 400 (or a remote electronic device) may also adjust or weight the blood pressure of the user 420 based on the position of the wearable blood pressure measurement device on the wearer of the wearable blood pressure measurement device.

In some examples, the second set of one or more sensor outputs may include a gravity angle generated by an IMU in the wearable blood pressure measurement device 402 (or sensor outputs from which the gravity angle may be determined). In these examples, a processor of the wearable blood pressure measurement device 402, or a processor of any of the electronic devices 400 (or a remote electronic device) that obtains the first set of one or more sensor outputs (or a blood pressure) and/or the second set of one or more sensor outputs from the wearable blood pressure measurement device 402, may use the gravity angle, as described with reference to FIGS. 2 and 3, to determine one or more conditions under which the first set of one or more sensor outputs is obtained. For example, the processor may determine at least one of a position or an orientation of the wearable blood pressure measurement device 402. The same processor, or a processor of another one of the electronic devices 400 (or a remote electronic device) may also adjust or weight the blood pressure of the user 420 based on the determined one or more conditions.

In some examples, the second set of one or more sensor outputs may include a first gravity angle generated by an IMU in the wearable blood pressure measurement device 402 (or sensor outputs from which the gravity angle may be determined) and a second gravity angle generated by an IMU in the electronic watch 404 or health/fitness tracking device (worn on the wrist of the arm on which the device 402 is worn) or the mobile phone 406 or other handheld portable device (held in the hand of the arm on which the device 402 is worn). In these examples, a processor of any of the electronic devices 400 (or a remote electronic device) that obtains the first set of one or more sensor outputs (or a blood pressure) and/or the second set of one or more sensor outputs from the wearable blood pressure measurement device 402 and electronic watch 404 or mobile phone 406, may use (and in some cases determine) the first and second gravity angles to determine a posing of the user's arm. In some cases, additional sensor outputs (e.g., emission and reception times of ultrasonic or optical pulses) may be used in combination with the first and second gravity angles to determine more detailed posing information for the arm. The same processor, or a processor of another one of the electronic devices 400 (or a remote electronic device) may also adjust or weight the blood pressure of the user 420 based on the determined posing of the arm and/or other determined conditions.

In some examples, the second set of one or more sensor outputs may include an orientation of a head-worn device (e.g., one or more earbuds 408 or a headset) worn by the user 420. In these examples, a processor of any of the electronic devices 400 (or a remote electronic device) that obtains the first set of one or more sensor outputs (or a blood pressure), and/or the second set of one or more sensor outputs, may use the orientation of the head-worn device to determine a head position of the user 420 and/or a posture or position of the user 420. The same processor, or a processor of another one of the electronic devices 400 (or a remote electronic device) may also adjust or weight the blood pressure of the user 420 based on the determined head position, posture, or position of the user 420.

In some examples, the second set of one or more sensor outputs may include a temperature of the user 420, a temperature of an ambient environment of the user 420, sounds suggesting particular activities of the user 420, a proximity of the user 420 to a particular device, a presence of the user 420 in a particular room, an indication of whether the user 420 is awake or asleep, and so on. A blood pressure may be adjusted or weighted in response to any or all of these sensor outputs.

In some cases, a collection of electronic devices, such as some or all of the electronic devices 400 shown in FIG. 4, may be used obtain sensor outputs that can be used to adjust, qualify, weight, or confirm measurements determined from other sensor outputs, or infer higher level parameters about a person. For example, a determination of an active calorie usage (or other health parameter, movement characteristic, or activity) can be improved with knowledge of a user's biophysical characteristics (e.g., height, weight, gait, and so on). Currently, an application may prompt a user to input these characteristics manually. However, one or more wearable, handheld, or semi-stationary electronic devices of the user may be able to obtain such biophysical characteristics and provide them to the application. For example, a wrist-worn of handheld electronic device may include an acoustic transducer or active optical sensor that generates sensor outputs usable to determine a length of a user's arm. As another example, a set of earbuds, or different earpieces of a headset, may include acoustic devices (e.g., acoustic emitters, acoustic receivers, or transducers) or optical devices (e.g., optical emitters, optical receivers, or active optical sensors), or a handheld electronic device may include a camera or depth sensor, that generate sensor outputs (e.g., time of flight measurements, images, or depth maps) that can be used to estimate a volume of a user's head or upper torso. In some cases, the volume of a user's head or upper torso may be used to estimate a user's body density. As another example, acoustic or optical reflectometry between a smart scale and earbuds or a headphone may generate sensor outputs that can be used to determine a user's height. As another example, a step counter or global positioning system (GPS) in a mobile phone, in combination with an IMU in an electronic watch, may generate sensor outputs that can be used to determine a user's gait (e.g., user positions over time, in combination with arm movements as the user moves). As another example, a user's movement, as characterized by IMU outputs obtained from an electronic device worn on one wrist, and a handheld device held in a hand attached to the opposite wrist, may be used to estimate the user's body volume.

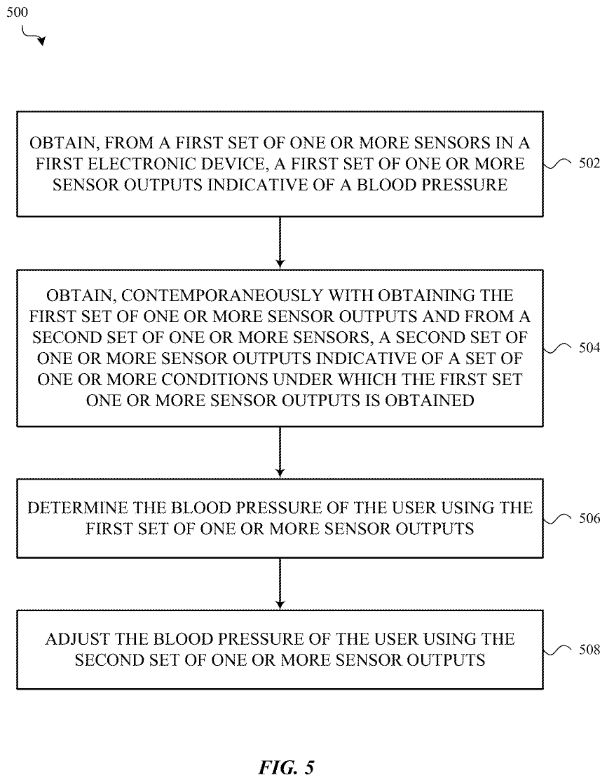

FIG. 5 shows an example method 500 of determining a blood pressure of a user. However, the method 500 may be used more generally to determine a health parameter of the user, or to characterize a movement or activity of the user.

At 502, the method 500 may include obtaining, from a first set of one or more sensors in a first electronic device, a first set of one or more sensor outputs indicative of a blood pressure (or other health parameter, or movement or activity) of the user. In some embodiments, the first electronic device may be a blood pressure measurement device.

At 504, the method 500 may include obtaining, contemporaneously with obtaining the first set of one or more sensor outputs and from a second set of one or more sensors, a second set of one or more sensor outputs indicative of a set of one or more conditions under which the first set one or more sensor outputs is obtained. In some embodiments, at least one sensor in the second set of one or more sensors may be included in a second electronic device, distinct from the first electronic device. In some embodiments, all of the sensors in the second set of one or more sensors may be included in the second electronic device and/or in a set of electronic devices that are distinct from the first electronic device. In some embodiments, at least one sensor in the second set of sensors, or all of the sensors in the second set of sensors, may be included in the first electronic device.

At 506, the method 500 may include determining the blood pressure (or other health parameter, or movement or activity) of the user using the first set of one or more sensor outputs.

At 508, the method 500 may include adjusting and/or weighting the blood pressure (or health parameter, or movement or activity) of the user using the second set of one or more sensor outputs.

In some embodiments of the method 500, adjusting the blood pressure of the user using the second set of one or more sensor outputs may include determining a position of a body part of the user, and adjusting the blood pressure of the user in response to the position of the body part of the user.

In some embodiments of the method 500, adjusting the blood pressure of the user using the second set of one or more sensor outputs may include determining a position of the first electronic device on a body part of the user, and adjusting the blood pressure of the user in response to the position of the first electronic device on the body part of the user.

In some embodiments of the method 500, adjusting the blood pressure of the user using the second set of one or more sensor outputs may include determining a movement or activity of the user, and adjusting the blood pressure of the user in response to the movement or activity of the user.

The blood pressure, movement, or activity of the user may be otherwise adjusted or weighted as described with reference to other figures.

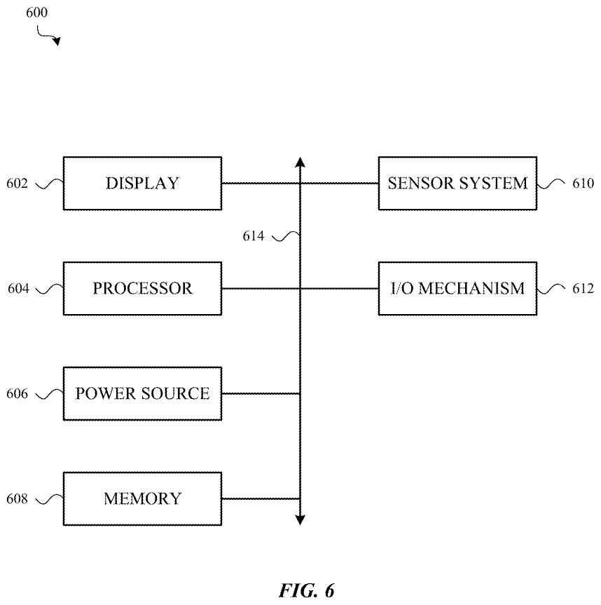

FIG. 6 shows an example electrical block diagram of an electronic device 600, such as one or more of the electronic devices described with reference to FIGS. 1-5. The electronic device 600 may include an optional display 602 (e.g., a light-emitting display), a processor 604, a power source 606, a memory 608 or storage device, a sensor system 610, and an optional input/output (I/O) mechanism 612 (e.g., an input/output device and/or input/output port). The processor 604 may control some or all of the operations of the electronic device 600. The processor 604 may communicate, either directly or indirectly, with substantially all of the components of the electronic device 600. For example, a system bus or other communication mechanism 614 may provide communication between the display 602, processor 604, the power source 606, the memory 608, the sensor system 610, and/or the input/output mechanism 612.

The processor 604 may be implemented as any electronic device capable of processing, receiving, or transmitting data or instructions. For example, the processor 604 may be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitably configured computing element or elements.

In some embodiments, the components of the electronic device 600 may be controlled by multiple processors. For example, select components of the electronic device 600 may be controlled by a first processor and other components of the electronic device 600 may be controlled by a second processor, where the first and second processors may or may not be in communication with each other.

The power source 606 may be implemented with any device capable of providing energy to the electronic device 600. For example, the power source 606 may include one or more disposable or rechargeable batteries. Additionally or alternatively, the power source 606 may include a power connector or power cord that connects the electronic device 600 to another power source, such as a wall outlet.

The memory 608 may store electronic data that may be used by the electronic device 600. For example, the memory 608 may store electrical data or content such as, for example, applications, device settings and user preferences, timing signals, control signals, data structures or databases, image data, sensor data, and so on. The memory 608 may be configured as any type of memory. By way of example only, the memory 608 may be implemented as random access memory, read-only memory, Flash memory, removable memory, other types of storage elements, or combinations of such devices.

The electronic device 600 may also include one or more sensors defining the sensor system 610. The sensors may be positioned substantially anywhere on the electronic device 600. The sensor(s) may be configured to sense substantially any type of characteristic, such as but not limited to, touch, force, pressure, sound, electromagnetic radiation (e.g., light), heat, movement, relative motion, biometric data, distance, and so on. For example, the sensor system 610 may include a touch sensor, a force sensor, a heat sensor, a position sensor, a light or optical sensor, a pressure sensor (e.g., a pressure transducer), an acoustic transducer, an IMU (e.g., an accelerometer or a gyroscope), a magnetometer, a health monitoring sensor, an image sensor, and so on. Additionally, the one or more sensors may utilize any suitable sensing technology, including, but not limited to, capacitive, ultrasonic, resistive, optical, ultrasound, piezoelectric, and thermal sensing technology.

The I/O mechanism 612 may transmit and/or receive data from a user or another electronic device. An I/O device may include a display, a touch sensing input surface such as a track pad, one or more buttons (e.g., a graphical user interface “home” button, or one of the buttons described herein), one or more cameras (including one or more image sensors), one or more microphones or speakers, one or more ports such as a microphone port, and/or a keyboard. Additionally or alternatively, an I/O device or port may transmit electronic signals via a communications network, such as a wireless and/or wired network connection. Examples of wireless and wired network connections include, but are not limited to, cellular, Wi-Fi, Bluetooth, IR, and Ethernet connections. The I/O mechanism 612 may also provide feedback (e.g., a haptic output) to a user.

The foregoing description, for purposes of explanation, uses specific nomenclature to provide a thorough understanding of the described embodiments. However, it will be apparent to one skilled in the art, after reading this description, that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art, after reading this description, that many modifications and variations are possible in view of the above teachings.

As described above, one aspect of the present technology may be the gathering and use of data available from various sources. The present disclosure contemplates that, in some instances, this gathered data may include personal information data (e.g., biological information) that uniquely identifies or can be used to identify, locate, contact, or diagnose a specific person. Such personal information data can include demographic data, location-based data, telephone numbers, email addresses, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to activate or deactivate various functions of the user's device, or gather performance metrics for the user's device or the user. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States (US), collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users may selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide mood-associated data for targeted content delivery services. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A wearable blood pressure measurement device, comprising:
a first set of one or more sensors;
a second set of one or more sensors; and
a processor configured to,
obtain, from the first set of one or more sensors, a first set of one or more sensor outputs indicative of a blood pressure of a wearer of the wearable blood pressure measurement device; and
obtain, contemporaneously with obtaining the first set of one or more sensor outputs and from the second set of one or more sensors, a second set of one or more sensor outputs indicative of a set of one or more conditions under which the first set one or more sensor outputs is obtained.

2. The wearable blood pressure measurement device of claim 1, wherein:
the processor is configured to,
determine the blood pressure of the wearer using the first set of one or more sensor outputs; and
adjust the blood pressure of the wearer using the second set of one or more sensor outputs.

3. The wearable blood pressure measurement device of claim 1, wherein:
the processor is configured to,
determine the blood pressure of the wearer using the first set of one or more sensor outputs; and
determine a confidence level for the blood pressure of the wearer using the second set of one or more sensor outputs.

4. The wearable blood pressure measurement device of claim 1, wherein:
the second set of one or more sensors includes a timer associated with at least one acoustic transducer;
obtaining the second set of one or more sensor outputs includes obtaining a time of emission of an ultrasonic pulse emitted by the at least one acoustic transducer and at least one time of receipt of at least one echo of the ultrasonic pulse; and
the processor is configured to,
cause the at least one acoustic transducer to emit the ultrasonic pulse and listen for the at least one echo of the ultrasonic pulse; and
determine, using the time of emission of the ultrasonic pulse and the at least one time of receipt of the at least one echo, a position of the blood pressure measurement device on the wearer of the wearable blood pressure measurement device.

5. The wearable blood pressure measurement device of claim 4, further comprising:
a blood pressure cuff; wherein,
each acoustic transducer in the at least one acoustic transducer has a known position with respect to the blood pressure cuff; and
the position of the blood pressure measurement device on the wearer of the blood pressure measurement device is a position of the blood pressure cuff on an arm of the wearer of the blood pressure measurement device.

6. The wearable blood pressure measurement device of claim 1, wherein:
the second set of one or more sensors includes a timer associated with at least one active optical sensor; and
obtaining the second set of one or more sensor outputs includes obtaining a time of emission of an optical pulse emitted by the at least one active optical sensor and at least one time of receipt of at least one reflection of the optical pulse; and
the processor is configured to,
cause the at least one active optical sensor to emit the optical pulse and monitor for the at least one reflection of the optical pulse; and
determine, using the time of emission of the optical pulse and the at least one time of receipt of the at least one reflection, a position of the blood pressure measurement device on the wearer of the wearable blood pressure measurement device.

7. The wearable blood pressure measurement device of claim 1, wherein:
the second set of one or more sensors includes an inertial measurement unit (IMU);
the second set of one or more sensor outputs includes a gravity angle; and
the processor is configured to,
determine, using an output of the IMU, the gravity angle; and
determine, using the gravity angle, at least one of: an orientation of the wearable blood pressure measurement device; a position or orientation of a body part on which the wearable blood pressure measurement device is worn; or a position or orientation of the wearable blood pressure measurement device with respect to a torso or heart of a wearer of the wearable blood pressure measurement device.

8. The wearable blood pressure measurement device of claim 1, further comprising:
a wireless communications interface; wherein,
the processor is configured to transmit, to a remote device,
at least one of the first set of one or more sensor outputs or the blood pressure; and
the second set of one or more sensor outputs.

9. An electronic device, comprising:
a wireless communications interface;
a processor configured to,
obtain a first set of one or more sensor outputs indicative of a blood pressure of a user;
obtain a second set of one or more sensor outputs indicative of a set of one or more conditions under which the first set one or more sensor outputs is obtained;
determine the blood pressure of the user using the first set of one or more sensor outputs; and
adjust or weight the blood pressure of the user using the second set of one or more sensor outputs;
wherein at least one sensor output of the first set of one or more sensor outputs or the second set of one or more sensor outputs is obtained via the wireless communications interface.

10. The electronic device of claim 9, wherein:
the first set of one or more sensor outputs is obtained from a wearable blood pressure measurement device worn on an arm of the user;
the second set of one or more sensor outputs include,
a time of emission of an ultrasonic pulse emitted by at least one acoustic transducer in the wearable blood pressure measurement device, and at least one time of receipt of at least one echo of the ultrasonic pulse; and
the processor is configured to,
determine, using the time of emission of the ultrasonic pulse and the at least one time of receipt of the at least one echo, a position of the wearable blood pressure measurement device on the wearer of the wearable blood pressure measurement device; and
adjust or weight the blood pressure of the user based on the position of the wearable blood pressure measurement device on the wearer of the wearable blood pressure measurement device.

11. The electronic device of claim 9, wherein:
the first set of one or more sensor outputs is obtained from a wearable blood pressure measurement device worn on an arm of the user;
the second set of one or more sensor outputs include,
a gravity angle generated by an inertial measurement unit (IMU) in the wearable blood pressure measurement device; and
the processor is configured to,
determine, using the gravity angle, at least one of a position or an orientation of the wearable blood pressure measurement device; and
adjust or weight the blood pressure of the user based on the at least one of the position or the orientation of the wearable blood pressure measurement device.

12. The electronic device of claim 9, wherein:
the first set of one or more sensor outputs is obtained from a wearable blood pressure measurement device worn on an arm of the user;
the second set of one or more sensor outputs include,
a first gravity angle generated by a first inertial measurement unit (IMU) in the wearable blood pressure measurement device; and
a second gravity angle generated by a second IMU in an electronic watch worn on a wrist of the arm of the user; and
the processor is configured to,
determine, using the first gravity angle and the second gravity angle, a posing of the arm; and
adjust or weight the blood pressure of the user based on the posing of the arm.

13. The electronic device of claim 12, wherein the electronic device is the electronic watch.

14. The electronic device of claim 9, wherein:
the first set of one or more sensor outputs is obtained from a wearable blood pressure measurement device worn on an arm of the user;
the second set of one or more sensor outputs include,
a first gravity angle generated by a first inertial measurement unit (IMU) in the wearable blood pressure measurement device; and
a second gravity angle generated by a second IMU in a portable device held by a hand of the arm of the user; and
the processor is configured to,
determine, using the first gravity angle and the second gravity angle, a posing of the arm; and
adjust or weight the blood pressure of the user based on the posing of the arm.

15. The electronic device of claim 14, wherein the electronic device is the portable device.

16. The electronic device of claim 9, wherein:
the second set of one or more sensor outputs include,
an orientation of a head-worn device worn by the user; and
the processor is configured to,
determine, using the orientation of the head-worn device, a head position of the user; and
adjust or weight the blood pressure of the user based on the head position of the user.

17. A method of determining a blood pressure, comprising:
obtaining, from a first set of one or more sensors in a first electronic device, a first set of one or more sensor outputs indicative of a blood pressure;
obtaining, contemporaneously with obtaining the first set of one or more sensor outputs and from a second set of one or more sensors, a second set of one or more sensor outputs indicative of a set of one or more conditions under which the first set one or more sensor outputs is obtained, at least one sensor in the second set of one or more sensors in a second electronic device;
determining the blood pressure of the user using the first set of one or more sensor outputs; and
adjusting the blood pressure of the user using the second set of one or more sensor outputs.

18. The method of claim 17, wherein adjusting the blood pressure of the user using the second set of one or more sensor outputs comprises:
determining a position of a body part of the user; and
adjusting the blood pressure of the user in response to the position of the body part of the user.

19. The method of claim 17, wherein adjusting the blood pressure of the user using the second set of one or more sensor outputs comprises:
determining a position of the first electronic device on a body part of the user; and
adjusting the blood pressure of the user in response to the position of the first electronic device on the body part of the user.

20. The method of claim 17, wherein adjusting the blood pressure of the user using the second set of one or more sensor outputs comprises:
determining a movement or activity of the user; and
adjusting the blood pressure of the user in response to the movement or activity of the user.
